(19) United States
(12) Patent Application Publication
(10) Pub. No.: US 2022/0213128 A1
(43) Pub. Date: Jul. 7, 2022

(54) L-Dopa Enhanced with a Neuroprotective Agent as a Therapy for Parkinson's Disease

(71) Applicant: RockGen Therapeutics LLC, Little Rock, AR (US)

(72) Inventors: Mahmoud Kiaei, Scarsdale, NY (US); Mohsen Sharifi, Indianapolis, IN (US)

(21) Appl. No.: 17/473,032

(22) Filed: Sep. 13, 2021

Related U.S. Application Data
(60) Provisional application No. 63/133,398, filed on Jan. 3, 2021.

(51) Int. Cl. C07F 7/08; C07D 339/04

(52) U.S. Cl. CPC C07F 7/0812; C07D 339/04

(57) ABSTRACT

Compounds and methods of using the compounds for the treatment of Parkinson's Disease are disclosed. The compound is created by the conjugation between L-Dopa and alpha lipoic acid.

### CROSS REFERENCE TO RELATED APPLICATIONS

This application claims priority to U.S. provisional patent application 63/133,398, filed on Jan. 3, 2021, and entitled “Development of L-Dopa enhanced with a neuroprotective agent as a therapy for Parkinson's Disease.” Such application is incorporated by reference herein in its entirety.

### STATEMENT REGARDING FEDERALLY SPONSORED RESEARCH OR DEVELOPMENT

Not applicable.

### BACKGROUND OF THE INVENTION

Parkinson's Disease (PD) is the second most common neurodegenerative disease worldwide and the number of people affected by PD is estimated to have more than doubled between 1990 and 2015. In the US, it is estimated that ˜1M new patients will be diagnosed with PD by 2020 and this will grow to 1.24M by 2030. The US anti-Parkinson's drugs market is predicted to grow from $1.1B to $1.4B in 2020 that will be higher year after year. As a chronic neurodegenerative disease, many PD patients may live 10-20 years after diagnosis and their symptoms progressively worsen. Despite intensive research, the etiology of PD remains unknown and currently available therapies only treat symptoms, not the underlying neurodegeneration. These symptomatic therapies help manage some of the symptoms early on but are ineffective at slowing disease progression. In addition to the obvious pain and physical burden to the patients and their families, PD costs the US $51.9 B every year, with $25.4B attributable to direct medical costs and $26.5 B in non-medical costs. Therefore, the growing population of patients, development of only symptomatic treatments, and the physical and economic burden indicate a significant need for a drug that can directly address neurodegeneration to halt or reverse PD.

The most recognizable PD symptoms are motor-related including tremor, bradykinesia (slowness of movement), rigidity, and postural instability. As PD progresses, neuropsychiatric disorders can also arise, including memory decline, dementia, and depression. These symptoms arise due to the loss of dopamine (DA)-producing neurons, which is the main pathological hallmark of PD. Therefore, replacing DA is a key strategy in improving PD symptoms. However, DA cannot cross the blood-brain barrier (BBB), but its precursor L-3,4-dihydroxyphenylalanine (L-Dopa) can. L-Dopa is the direct precursor to DA and still is the most effective pharmacological therapy for the treatment of motor symptoms in PD. In DA-producing neurons, L-Dopa is synthesized from tyrosine by tyrosine hydroxylase (TH), then L-Dopa is converted to DA by a decarboxylase enzyme. These neurons are the main source of DA in the central nervous system, which is necessary for many basic brain functions ranging from motor control to behavior.

The problem is that L-Dopa (as Levodopa or Sinemet®) has many disadvantages as a PD therapeutic. First, most of L-Dopa can be prematurely converted to DA by enzymes in the peripheral tissue and the microbiome can metabolize L-Dopa in the gut, rendering the bioavailability of L-Dopa to just 1%. One way to improve L-Dopa bioavailability is to deliver L-Dopa with carboxylase inhibitor, carbidopa, but this is also associated with significant adverse side-effects. Second, as a chronic disease, PD requires long-term treatment with L-Dopa, which causes adverse side-effects and potentially worsen PD symptoms through oxidative stress and inflammation. Third, in the first few years of PD, since the majority of cells that produce dopamine are still alive, L-Dopa does help with symptoms, but, its effectiveness wears-off as the disease inevitably progresses, therefore when more cells die, the dopamine levels decline further and the disease worsens in PD patients.

Another fundamental problem that remains to be addressed is that L-Dopa requires decarboxylase enzymes to synthesize DA, which declines in the striatum with disease progression. L-Dopa's bioavailability is drastically reduced due to degradation and premature metabolism in peripheral tissues (half-life of 0.5 h) and, with certain formulations, only 5-10% L-Dopa of oral dose may reach the brain at best. Even with this low bioavailability, L-Dopa can relieve some symptoms in the early stages of PD and is the most prescribed therapeutic for PD. On the other hand, various formulations of L-Dopa are only symptomatic treatment without any disease modification and do not address the underlying pathology of neurodegeneration in PD. Oxidative stress is thought to contribute to the death of the dopaminergic neurons and antioxidants were tested for treating PD, but studies with vitamin E, Coenzyme Q (CoQ), and creatine have proven ineffective.

Other PD pharmacologic treatments also focus on increasing DA levels by using DA agonists or inhibitors of enzymes that metabolize DA. As recent as 2019, the FDA approved Nourianz (blocker of the adenosine A receptor) as an “add on” to levodopa/carbidopa for PD patients experiencing “off” episodes. Clinical evaluations with Nourianz® showed symptomatic relief, without any disease modification. In another option, deep brain stimulation is a surgical procedure that can be used when patients have unstable medication responses. However, these current methods have significant limitations that only relieve symptoms in PD, presenting a significant need for a treatment that can halt or delay PD progression.

Given all these problems with existing technologies and treatment options, the development of a new drug with better clinical efficacy, fewer side effects, and neuroprotective activity is an important unmet medical need for PD. We aim to address this issue by delivering an antioxidant that chemically bonded with L-Dopa protects neurons expressing decarboxylase and synthesizing DA.

Substantial and convincing evidence published over the years has shown that PD is linked to excess production of reactive oxygen species (ROS) leading to oxidative stress and neuronal death associated with neurodegeneration due to mitochondrial dysfunction, neuroinflammation, and DA metabolism. After DA is produced, it is stored in synaptic vesicles under normal conditions. However, when there is excess DA than the vesicle's capacity to uptake, with each L-Dopa dosing, cytosolic DA metabolism results in cytotoxic ROS by auto-oxidation or monoamine oxidase (MAO). One approach to protect neurons in PD is by effectively scavenging the ROS.

Alpha lipoic acid (ALA) is a naturally occurring 8-carbon fatty acid (FA) that is synthesized de novo from octanoic acid in mitochondria in plants and animals. It is a naturally occurring molecule in humans and the body knows how to use it for its advantage and has all the necessary machinery for its metabolism, hence the possibility of adverse effects and toxicity is rather low. ALA is a potent antioxidant that reduces and scavenge 7 radical species including ROS, stimulates the activation and upregulation of other antioxidants including glutathione, and chelates redox-active metals. Neuronal plasma and axonal membranes are rich in polyunsaturated FAs with an inherent lower antioxidant ability. Thiols are central to antioxidant defense for neurons and mostly come from glutathione, which cannot be directly administered. ALA's thiol is an alternative. ALA is used in several diseases associated with oxidative stress, which is significantly effective with no serious side-effects at moderate doses. Prior studies show ALA protects neurons against oxidative stress-induced death in in vivo and in vitro models of neurodegenerative diseases, including PD. Further, ALA exerts anti-inflammatory effects by down-regulating the expression of redox-sensitive pro-inflammatory proteins including TNF and inducible nitric oxide synthase.

The inventors hereof have determined that conjugating a potent antioxidant, such as ALA, to L-Dopa allows for the delivery of a chemically bonded compound serving as a substrate for DA synthesis and attenuate oxidative stress in PD.

### BRIEF SUMMARY OF THE INVENTION

This invention relates to a drug platform for use in the treatment of Parkinson's Disease (PD), where the platform provides better clinical efficacy, causes fewer side effects, and provides neuroprotective activity. The drug platform consists of a number of compounds created by the conjugation between L-Dopa and ALA. With the conjugation of the fatty acid ALA to the dopamine precursor L-Dopa, drug delivery is enhanced and brain targeting is improved.

In one embodiment, the present invention is directed to the methodology for conjugating antioxidant ALA directly to L-Dopa to provide novel compounds for the treatment of PD. The benefits of the conjugation of ALA directly to L-Dopa are two-fold. First, it provides improved delivery of intermediate substrate to the brain to synthesize dopamine (DA), and second, it prevents further neurodegeneration by protecting neurons from oxidative stress. A proprietary computer-aided drug discovery technique (including fragment/structure-based drug design) is used to design over one hundred L-Dopa-ALA conjugates, and predictive computational modeling is used to evaluate parameters such as human oral absorption, membrane permeability, and drug-likeness to determine which compounds are the best candidates for effectively treating PD. Four candidates, which are referred to herein as RG-1, RG-2, RG-3, and RG-4, have been found to be the most stable compounds and the compounds having the most favorable characteristics for treatment of PD. One compound, in particular, RG-1, has been found to have a superior drug profile and is predicted to become the most successful as an orally active drug. In any event, it has been found that the compounds of the present invention (including all of RG-1, RG-2, RG-3, and RG-4) are synthesizable and appropriate as brain-targeting drugs and are successful in protecting DA synthesizing neurons, making these novel compounds beneficial for the treatment of PD.

Currently available treatments for PD only treat symptoms. Furthermore, these therapeutics have poor bioavailability, adverse side-effects, and may hasten PD symptoms. There is currently no approved therapy that successfully goes beyond symptoms and prevents neurodegeneration. The present invention provides an innovative strategy by taking advantage of machine learning for the development of a drug platform employing lipid-drug conjugation chemistry to develop a novel drug to halt or delay further neurodegeneration in PD. This innovation is superior and has significant value over currently available therapies as previously direct and single administered antioxidants (Vitamin E, CoQ, creatine, etc.) have failed in the clinic, likely due to delivery to the target tissue. Direct conjugation of ALA, an antioxidant compound to L-Dopa, as provided in the present invention increases L-Dopa reaching the brain, increases bioavailability in dopaminergic neurons, and protects the dopaminergic cells from death.

The lipid-drug conjugates of the present invention offer the power to avoid premature hydrolysis and exhibit increased interactions with cell membranes, which both contribute to increased bioavailability, improved brain targeting, and better BBB penetration. The present invention chemically joins lipid and drug to overcome the limitations of current treatments using proprietary techniques to synthesize a novel therapeutic to be evaluated for bioavailability and neuroprotective effects and mitigation of adverse side-effects. Conventional drug discovery via high throughput screening has been the standard for drug discovery programs. However, this method can take up to 12-15 years to progress from discovery to market and potentially cost upwards of $1B. The method of the present invention expedites the discovery timeline by using a unique approach to design L-Dopa-ALA compounds through computer-aided drug design and laboratory testing.

The compounds of the present invention are a novel therapeutic for the treatment of PD, and the method for producing the compounds, provides a novel approach for the production of such innovative drugs. These and other objects, features, and advantages of the present invention will become better understood from a consideration of the following detailed description of the preferred embodiments and appended claims in conjunction with the drawings as described following:

### BRIEF DESCRIPTION OF THE DRAWINGS

FIG. 1A shows one embodiment of the chemical structure of the compound for the treatment of PD of the present invention, where A, C, D and E are sites for modifications to add side atoms/group(s) to the ALA portion of the compound. In this embodiment, ALA is conjugated to L-Dopa at the X position (or IPSO position). FIG. 1B shows an alternative embodiment of the chemical structure of the compound for the treatment of PD of the present invention with ALA conjugated to L-Dopa at the Y position (or ORTHO position).

FIG. 2 shows one embodiment of the chemical structure and synthesis scheme for RG-1.

FIG. 3 shows one embodiment of the chemical structure and synthesis scheme for RG-2.

FIG. 4 shows one embodiment of the chemical structure and synthesis scheme for RG-3.

FIG. 5 shows one embodiment of the chemical structure and synthesis scheme for RG-4.

FIG. 6 shows results of ADME/tox in-silico analysis of RG-1 and levodopa using Schrodinger software.

FIG. 7 shows results of ADME/tox in-silico analysis of RG-1 and levodopa using ChemAxon software.

FIG. 8 shows results of an in-silico docking simulation of RG-1 in the dopa-decarboxylase binding pocket.

FIG. 9 shows results of binding scores by molecular docking poses of RG-1 and control compounds or target binding validation.

FIG. 10A shows RG-1 interacting with FATP in a docking experiment at pose 1 of the same binding pocket.

FIG. 10B shows RG-1 interacting with FATP in a docking experiment at pose 2 of the same binding pocket.

FIG. 10C shows RG-1 interacting with FATP in a docking experiment at pose 3 of the same binding pocket.

FIG. 11 shows RG-1 in a docking experiment with decarboxylase.

FIG. 12 shows RG-1 interacting with α-syn at the glycerol binding site in a docking experiment.

FIG. 13 shows the chemical structure of RG-1 with conjugation of ALA to L-Dopa at the IPSO position.

FIG. 14 shows the chemical structure of RG-1 with conjugation of ALA to L-Dopa at the ORTHO position.

FIG. 15 shows the chemical structure of RG-2 with conjugation of ALA to L-Dopa at the IPSO position.

FIG. 16 shows the chemical structure of RG-2 with conjugation of ALA to L-Dopa at the ORTH position.

FIG. 17 shows the chemical structure of RG-3 with conjugation of ALA to L-Dopa at the IPSO position.

FIG. 18 shows the chemical structure of RG-3 with conjugation of ALA to L-Dopa at the ORTHO position.

FIG. 19 shows the chemical structure of RG-4 with conjugation of ALA to L-Dopa at the IPSO position.

FIG. 20 shows the chemical structure of RG-4 with conjugation of ALA to L-Dopa at the ORTHO position.

FIG. 21A shows the chemical structure of one embodiment of the R group of the compound of the present invention. FIG. 21B shows the chemical structure of one embodiment of the R group of the compound of the present invention. FIG. 21C shows the chemical structure of one embodiment of the R group of the compound of the present invention. FIG. 21D shows the chemical structure of one embodiment of the R group of the compound of the present invention.

### DETAILED DESCRIPTION OF THE INVENTION

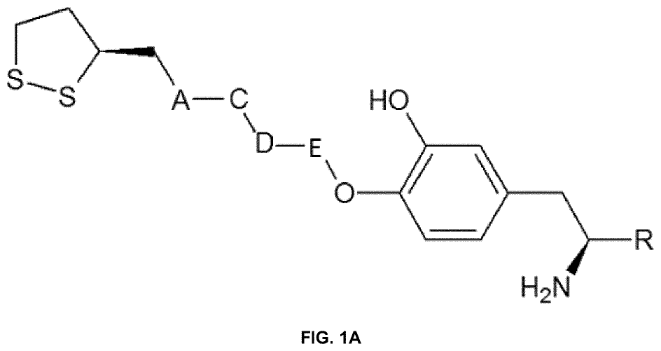

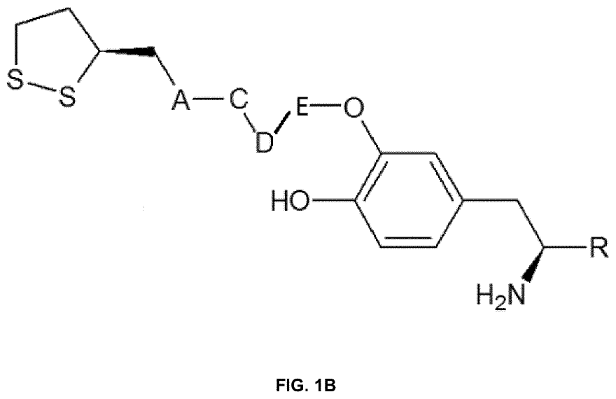

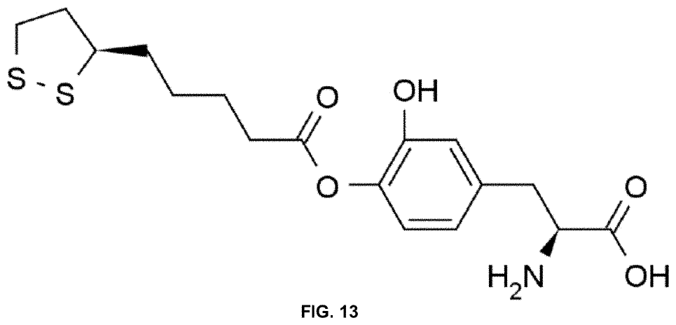

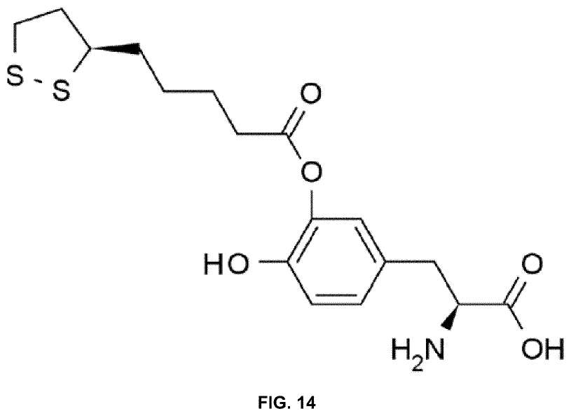

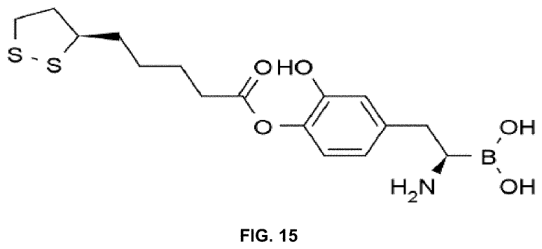

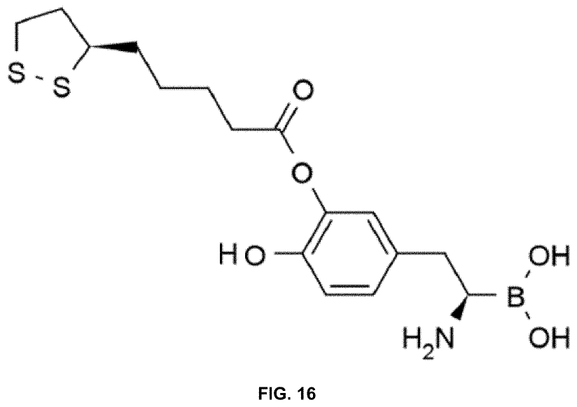

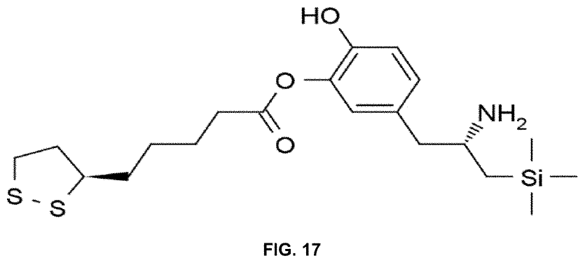

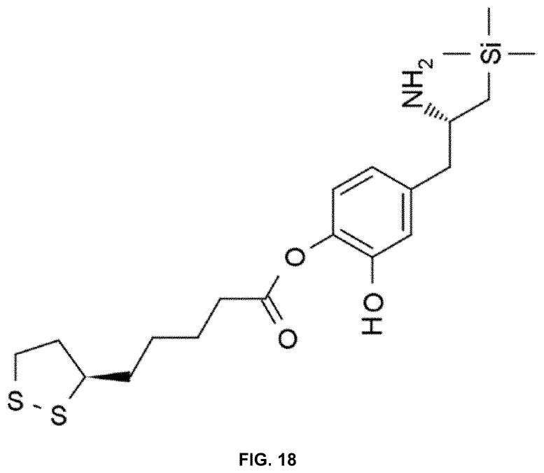

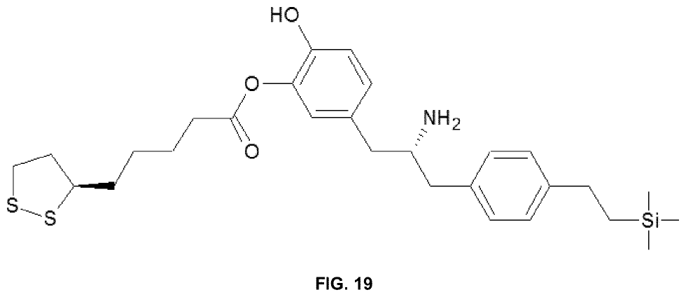

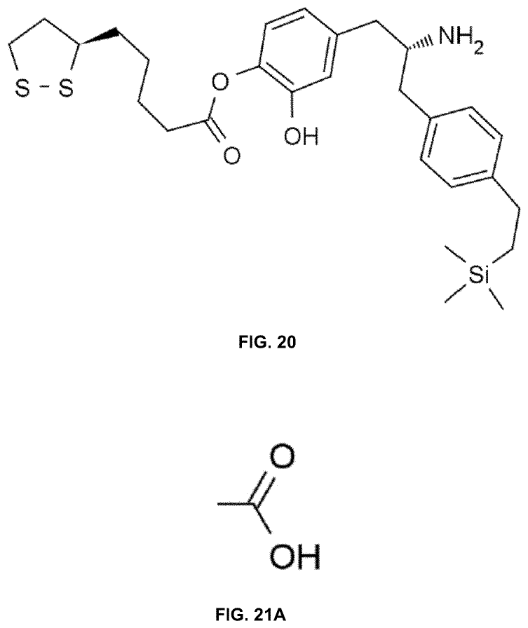

With reference to FIGS. 1A-21D, the preferred embodiments of the present invention may be described. Generally speaking, the present invention is directed to a drug platform for use in the treatment of motor neuron disease (MND), multiple sclerosis (MS), and Parkinson's Disease (PD), where the platform provides better clinical efficacy, causes fewer side effects, and provides neuroprotective activity. The compounds may also be used for the treatment of Parkinson-Dementia-Complex and other neurodegenerative diseases and neurological disorders. The drug platform consists of a number of compounds created by the conjugation between L-3,4-dihydroxyphenylalanine (L-dopa) and alpha lipoic acid (ALA). While the proprietary method of the present invention for conjugating lipids and L-dopa to provide PD-effective compounds (as described more fully below) provides for the creation of numerous compounds, the preferred compounds for use in the treatment of PD are referred to herein as RG-1, RG-2, RG-3, and RG-4. Particularly, these multifunctional co-drugs conjugate with antioxidant properties to protect neuro-dopaminergic neurons, and the invention relates to treating subjects with a pharmaceutically acceptable dose of compounds, crystals, esters, salts, hydrates, prodrugs, or mixtures thereof. Pharmaceutically acceptable salt or ester thereof, are used for treating or preventing diseases, disorders, or conditions related to oxidative stress, and/or to prevent or treat stroke, ischemia, reperfusion injury, or combinations thereof.

Specific examples of the diseases, disorders, or conditions related to oxidative stress include, but are not limited to, stroke, ischemia, reperfusion injury, or combinations thereof. The compounds or compositions described herein can be suitably formulated into one or more than one separate pharmaceutical compositions for administration to subjects in a biologically compatible form suitable for administration in vivo. Accordingly, the present application also includes a pharmaceutical composition comprising one or more compounds or compositions and a pharmaceutically acceptable carrier.

### The Compounds

The present invention is directed to a compound of Formula I:

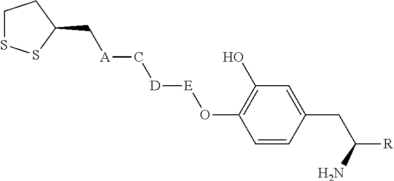

or a salt thereof, wherein:

A is chosen from methyl, ethyl, ethane, hydroxyl group, para chlorobenzene, benzenethiol, toluene, 4-methylphenol, phenyl, methanol, ethanol, methanol, ammonia, fluoromethane, nitrous acid, hydroxylamine, hydroxy(methyl)oxoammonium, trifluoromethane, chloromethane, chloride, sulphur, methanesulfinic acid, methanamine, ethanamine, nitromethane, hydroxylamine, and acetic acid;

C is chosen from methyl, ethyl, ethane, carboxylic acid, hydroxyl group, para chlorobenzene, benzenethiol, toluene, 4-methylphenol, phenyl, methanol, ammonia, fluoromethane, trifluoroethane, nitrous acid, hydroxylamine, hydroxy(methyl)oxoammonium, chloromethane, chloride, sulphur, methanesulfinic acid, acetic acid, methanol, methanethiol, ethanol, ethenol, methanamine, and hydroxylamine;

D is chosen from methyl, ethyl, and methanethiol;

E is chosen from formaldehyde and ethyl; and

R is chosen from:

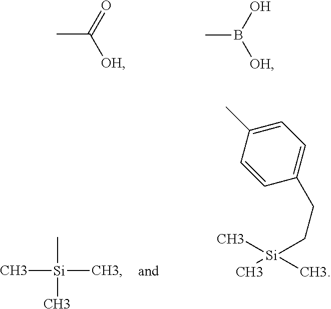

In one embodiment, A is methyl, C is methyl, D is methyl, E is formaldehyde, and R is:

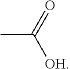

In one embodiment, A is methyl, C is methyl, D is methyl, E is formaldehyde, and R is:

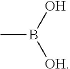

In one embodiment, wherein A is methyl, C is methyl, D is methyl, E is formaldehyde, and R is:

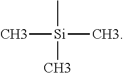

In one embodiment, A is methyl, C is methyl, D is methyl, E is formaldehyde, and R is:

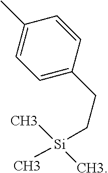

The present invention is also directed to a compound of Formula I:

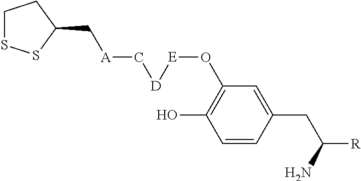

or a salt thereof, wherein:

A is chosen from methyl, ethyl, ethane, hydroxyl group, para chlorobenzene, benzenethiol, toluene, 4-methylphenol, phenyl, methanol, ethanol, methanol, ammonia, fluoromethane, nitrous acid, hydroxylamine, hydroxy(methyl)oxoammonium, trifluoromethane, chloromethane, chloride, sulphur, methanesulfinic acid, methanamine, ethanamine, nitromethane, hydroxylamine, and acetic acid; C is chosen from methyl, Ethyl, ethane, carboxylic acid, hydroxyl group, para chlorobenzene, benzenethiol, toluene, 4-methylphenol, phenyl, methanol, ammonia, fluoromethane, trifluoroethane, nitrous acid, hydroxylamine, hydroxy(methyl)oxoammonium, chloromethane, chloride, sulphur, methanesulfinic acid, acetic acid, methanol, methanethiol, ethanol, ethenol, methanamine, and hydroxylamine;

D is chosen from methyl, ethyl, and methanethiol;

E is chosen from formaldehyde and ethyl; and

R is chosen from:

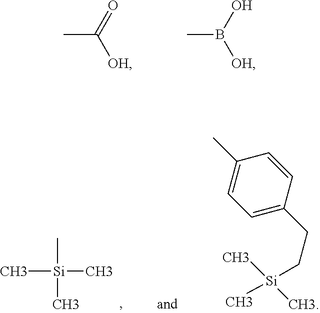

In one embodiment, A is methyl, C is methyl, D is methyl, E is formaldehyde, and R is:

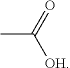

In one embodiment, A is methyl, C is methyl, D is methyl, E is formaldehyde, and R is:

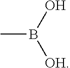

In one embodiment, A is methyl, C is methyl, D is methyl, E is formaldehyde, and R is:

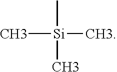

In one embodiment, A is methyl, C is methyl, D is methyl, E is formaldehyde, and R is:

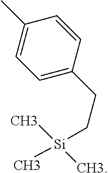

The present invention is also directed to a method for treating Parkinson's Disease in a subject in need thereof, comprising administering to the subject a therapeutically effective amount of either of the following compounds:

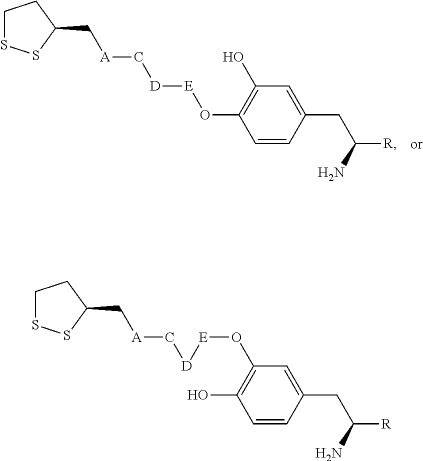

or a salt thereof, wherein:

A is chosen from methyl, ethyl, ethane, hydroxyl group, para chlorobenzene, benzenethiol, toluene, 4-methylphenol, phenyl, methanol, ethanol, methanol, ammonia, fluoromethane, nitrous acid, hydroxylamine, hydroxy(methyl)oxoammonium, trifluoromethane, chloromethane, chloride, sulphur, methanesulfinic acid, methanamine, ethanamine, nitromethane, hydroxylamine, and acetic acid; C is chosen from methyl, Ethyl, ethane, carboxylic acid, hydroxyl group, para chlorobenzene, benzenethiol, toluene, 4-methylphenol, phenyl, methanol, ammonia, fluoromethane, trifluoroethane, nitrous acid, hydroxylamine, hydroxy(methyl)oxoammonium, chloromethane, chloride, sulphur, methanesulfinic acid, acetic acid, methanol, methanethiol, ethanol, ethenol, methanamine, and hydroxylamine;

D is chosen from methyl, ethyl, and methanethiol;

E is chosen from formaldehyde and ethyl; and

R is chosen from:

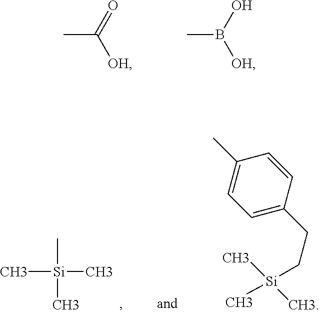

In one embodiment of the method, A is methyl, C is methyl, D is methyl, E is formaldehyde, and R is:

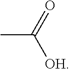

In one embodiment of the method, A is methyl, C is methyl, D is methyl, E is formaldehyde, and R is:

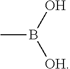

In one embodiment of the method, A is methyl, C is methyl, D is methyl, E is formaldehyde, and R is:

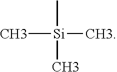

In one embodiment of the method, A is methyl, C is methyl, D is methyl, E is formaldehyde, and R is:

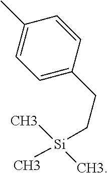

In one embodiment, the present invention is directed to one or more compounds that are useful in the treatment of PD. Generally speaking, the present invention encompasses a series (over 100 compounds) of L-Dopa conjugates utilizing FA scaffold-type compounds and metalloids to develop analogs as lead compounds to be developed as an alternative to Levodopa. And in one particular implementation, the compounds are created by the conjugation of L-Dopa and ALA. ALA is chemically attached to L-Dopa to assist in the delivery of L-Dopa to neurons that L-Dopa enters de novo. This allows for the reduction of the “L-Dopa off time” because L-Dopa is slowly released from ALA and also allows for the potential to avoid peripheral premature metabolism. The developed compounds act as a combined L-dopa and carrier-mediated transporter that use a combined strategy to improve BBB penetration and some other drug delivery properties, with slow and sustained release capability which reduces plasma fluctuation. The compounds also protect dopaminergic neurons due to the antioxidant property of ALA.

While the present invention encompasses hundreds of compounds that are potentially useful in the treatment of PD, there are four primary compounds (RG-1, RG-2, RG-3, and RG-4) that have been found to be particularly suitable for the treatment of PD. As stated earlier, in PD, some brain cells that produce dopamine (DA-producing neurons) gradually die. To compensate for the dopamine loss, PD patients take a medication called Levodopa, which is a pro-drug of dopamine and actually converts to dopamine in brain. However, when disease progress and more brain cells die, dopamine concentration from Levodopa medication is not be enough. RG-1 is designed to bind to the transporters in gut, so it will be transferred easier into body. It can then bind to other transporters to pass BBB to reach the brain. Once in the brain, it breaks down (hydrolyzed) into dopamine and an amino acid. The amino acid (i.e. lipoic acid, ALA) portion is a safe antioxidant in humans and also has some neuroprotective effects. RG-1 can be also considered as a “me-better” (improved and better than the current medication, e.g. Levodopa/carbidopa) or “best-in-class” drugs for PD that can deliver enough dopamine while protecting remaining brain cells.

The general chemical structure of the compounds of the present invention is shown in FIGS. 1A-1B, where A, C, D and, E are selected from the atom(s) and group(s) listed in the Table below:

| | Positions | | | |
|---|---|---|---|---|
| | A | C | D | E |
| 1 | —CH2CH3 | —CH3 | —CH3 | —C═O |
| 2 | —CH3 | —CH3 | —CH3 | —CH2CH3 |
| 3 | —CH3 | —CH2CH3 | —CH3 | —C═O |
| 4 | —CH3 | —CH3 | —CH2CH3 | —C═O |
| 5 | —CH2CH3 | —CH2CH3 | —CH3 | —C═O |
| 6 | —CH2CH3 | —CH3 | —CH2CH3 | —C═O |
| 7 | —CH3 | —CH2CH3 | —CH2CH3 | —C═O |
| 8 | —CH2CH3 | —CH2CH3 | —CH2CH3 | —C═O |
| 9 | p-Cl—Ph— | —CH3 | —CH2CH3 | —C═O |
| 10 | —CH3 | p-Cl—Ph— | —CH3 | —C═O |
| 11 | p-SH—Ph— | —CH3 | —CH3 | —C═O |
| 12 | —CH3 | p-SH—Ph— | —CH3 | —C═O |
| 13 | Ph— | —CH3 | —CH3 | —C═O |
| 14 | —C—Ph | —CH3 | —CH3 | —C═O |
| 15 | —C—Ph | —CH2CH3 | —CH3 | —C═O |
| 16 | —C—Ph—OH | —CH2CH3 | —CH3 | —C═O |
| 17 | —C—Ph—OH | —CH3 | —CH3 | —C═O |
| 18 | —CH3 | Ph— | —CH3 | —C═O |
| 19 | —CH3 | C—Ph— | —CH3 | —C═O |
| 20 | —CH3 | —C—Ph—OH | —CH3 | —C═O |
| 21 | —CH2CH3 | C—Ph— | —CH3 | —C═O |
| 22 | —O—CH3 | —CH3 | —CH3 | —C═O |
| 23 | —NH2 | —CH3 | —CH3 | —C═O |
| 24 | —C—CF3 | —CH3 | —CH3 | —C═O |
| 25 | —CF | —CH3 | —CH3 | —C═O |
| 26 | —C—Cl | —CH3 | —CH3 | —C═O |
| 27 | —Cl | —CH3 | —CH3 | —C═O |
| 28 | —SH | —CH3 | —CH3 | —C═O |
| 29 | —CHO | —CH3 | —CH3 | —C═O |
| 30 | —CCHO | —CH3 | —CH3 | —C═O |
| 31 | —OH | —CH3 | —CH3 | —C═O |
| 32 | —COH | —CH3 | —CH3 | —C═O |
| 33 | —CN | —CH3 | —CH3 | —C═O |
| 34 | —C—CN | —CH3 | —CH3 | —C═O |
| 35 | —NO2 | —CH3 | —CH3 | —C═O |
| 36 | —C—NO2 | —CH3 | —CH3 | —C═O |
| 37 | —NO | —CH3 | —CH3 | —C═O |
| 38 | —C—NO | —CH3 | —CH3 | —C═O |
| 39 | —CH═CH2 | —CH3 | —CH3 | —C═O |
| 40 | —CH3 | —O—CH3 | —CH3 | —C═O |
| 41 | —CH3 | —NH2 | —CH3 | —C═O |
| 42 | —CH3 | —C—CF3 | —CH3 | —C═O |
| 43 | —CH3 | —CF | —CH3 | —C═O |
| 44 | —CH3 | —Cl | —CH3 | —C═O |
| 45 | —CH3 | —C—Cl | —CH3 | —C═O |
| 46 | —CH3 | —C—COOH | —CH3 | —C═O |
| 47 | —C—COOH | —CH3 | —CH3 | —C═O |
| 48 | —CH3 | —COOH | —CH3 | —C═O |
| 49 | —CH3 | —C—OH | —CH3 | —C═O |
| 50 | —CH3 | —OH | —CH3 | —C═O |
| 51 | —CH3 | —CH═CH2 | —CH3 | —C═O |
| 52 | —CH3 | —C—SH | —CH3 | —C═O |
| 53 | —CH3 | —SH | —CH3 | —C═O |
| 54 | —CH3 | —COH | —CH3 | —C═O |
| 55 | —CH3 | —C—COH | —CH3 | —C═O |
| 56 | —CH3 | —C═COH | —CH3 | —C═O |
| 57 | —CH3 | —CN | —CH3 | —C═O |
| 58 | —CH3 | —NO2 | —CH3 | —C═O |
| 59 | —CH3 | —NO | —CH3 | —C═O |
| 60 | —CH3 | —C—NO2 | —CH3 | —C═O |
| 61 | —CH3 | —C—NO | —CH3 | —C═O |
| 62 | —CH3 | —C—NO2 | —CH2CH3 | —C═O |
| 63 | —CH3 | —C—NO | —CH2CH3 | —C═O |
| 64 | —CH3 | —CH3 | —C—SH | —C═O |
| 65 | —CH2CH3 | —CH3 | —C—SH | —C═O |
| 66 | —CH3 | —CH2CH3 | —C—SH | —C═O |
| 67 | —CH2CH3 | —CH2CH3 | —C—SH | —C═O |
| 68 | —CH3 | —C—SO2 | —CH3 | —C═O |
| 69 | —C—SO2 | —CH3 | —CH3 | —C═O |

In the Table, Ph stands for phenyl and p- is para location. Other side chain and groups are well defend groups are: CH3=methyl, CH2CH3=Ethyl, CH═CH2=ethane, COOH=carboxylic acid, OH=hydroxyl group, p-Cl-Ph-=para chlorobenzene, p-SH-Ph-=benzenethiol, C-Ph=toluene, C-Ph-OH=4-methylphenol, Ph-=phenyl, C—OH=methanol, CCHO=ethanol, OCH3=methoxy, NH2=ammonia, CF=fluoromethane, C—CF3=trifluoroethane, NO2=nitrous acid (also known as nitrogen dioxide), C—NO=hydroxylamine, C—NO2=hydroxy(methyl)oxoammonium, CF3=trifluoromethane, C—Cl=chloromethane, Cl=chloride, SH=Sulphur, C—SO2=methanesulfinic acid, CN=methanamine, C—CN=ethanamine, C—NO2=nitromethane, NO=hydroxylamine, C—COOH=acetic acid, C—SH=methanethiol, C—COH=ethanol, C═COH=ethenol, and C═O=formaldehyde.

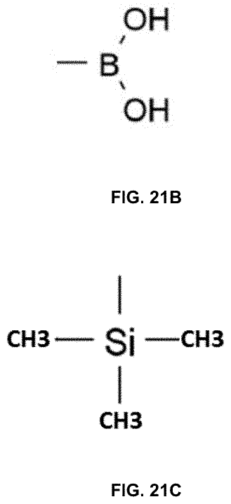

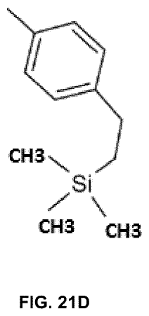

The R group may be chosen from the group consisting of R1 (shown in FIG. 21A), R2 (shown in FIG. 21B), R3 (shown in FIG. 21C), and R4 (shown in FIG. 21D).

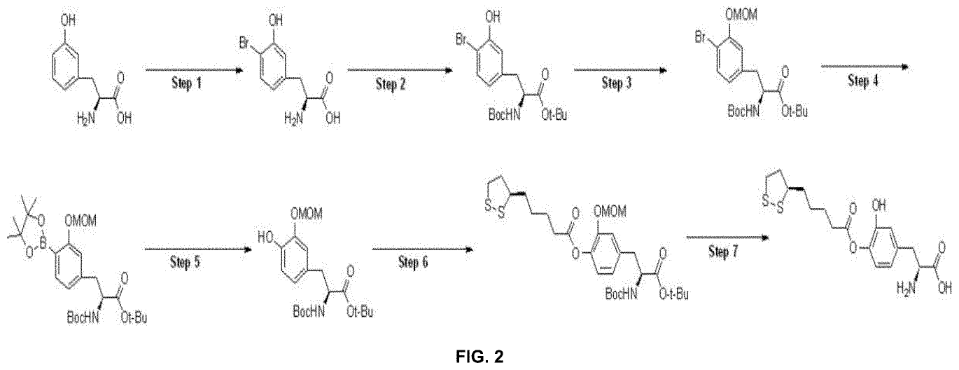

The structure and synthesis scheme for each of these leading compounds are shown in FIGS. 2-5. In particular, FIG. 2 shows the first leading compound (referred to as RG-1), and the synthesis scheme for the same.

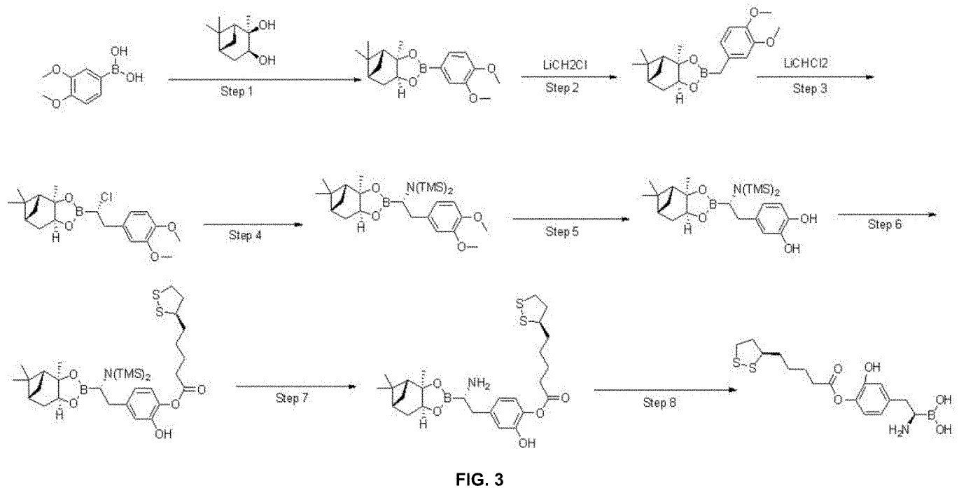

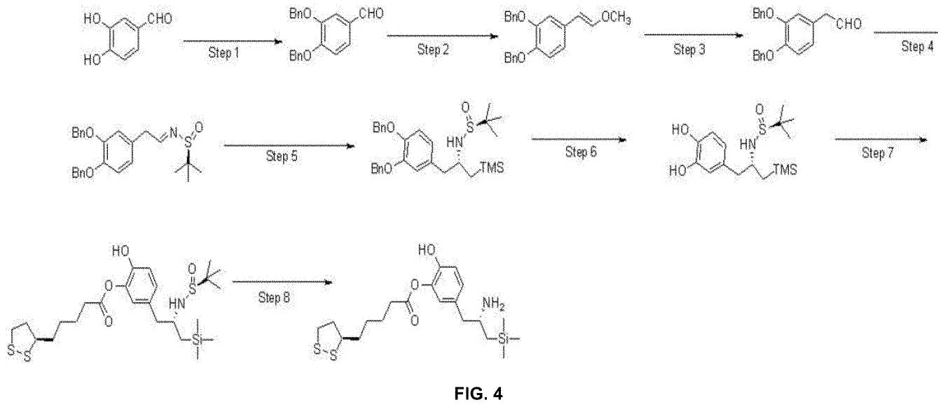

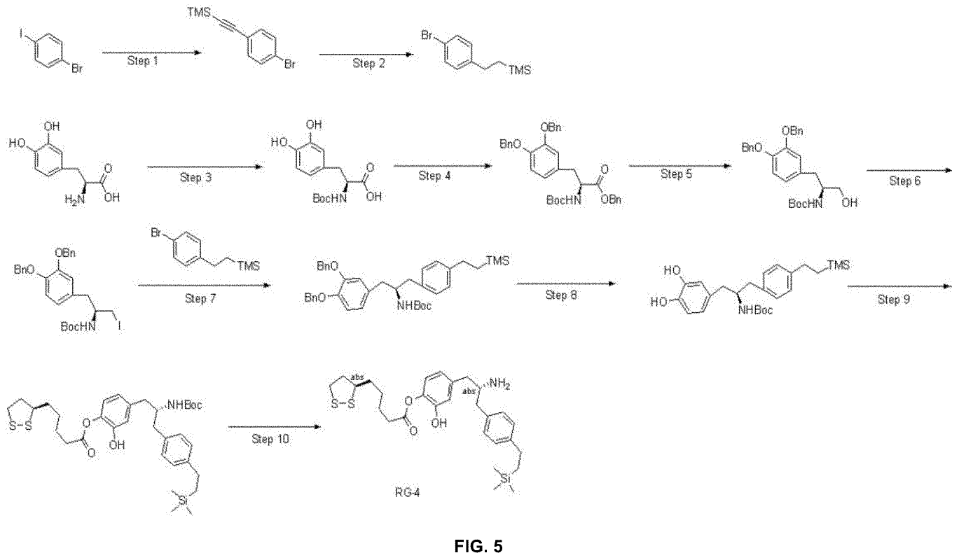

FIG. 3 shows the second leading compound (referred to as RG-2), which is modified with Boron, and the synthesis scheme for the same. FIG. 4 shows the third leading compound (referred to as RG-3), which has been modified with Silicon, and the synthesis scheme for the same. Finally, FIG. 5 shows the fourth leading compound (referred to as RG-4), which has been modified with a Silicon bonded benzene ring, and the synthesis scheme for the same. The synthesis of the compounds shown in FIGS. 2-5 was measured by 1) 1H NMR; 2) LC/MS with a run time of longer than 2 minutes; and 3) HPLC UV 214 nm and 254 nm, and only purities higher than 97% were considered. The above Table describes the chemical structure of RG-1, RG-2, RG-3 and RG-4. In FIGS. 4-5 and 17-20, the three lines extending from “Si” indicate a bond with “CH3,” as would be understood by a person of ordinary skill in the art.

The compounds or compositions may be administered to a subject in a variety of forms depending on the selected route of administration, as will be understood by those skilled in the art. A compound may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time. Conventional procedures and ingredients for the selection and preparation of suitable compositions are described, for example, in Remington's Pharmaceutical Sciences (2000-20^{th} edition) and in the United States Pharmacopeia: The National Formulary (USP 24 NF19) published in 1999.

The compounds or compositions may be orally administered, for example, with inert diluents or with an assimilable edible carrier, or the compounds may be enclosed in hard or soft shell gelatin capsules, compressed into tablets, or incorporated directly with the food of the diet. For oral therapeutic administration, the compound may be incorporated with excipient and used in the form of ingestible tablets, buccal tablets, troches, capsules, elixirs, suspensions, syrups, wafers, and the like. Oral dosage forms also include modified release, for example, immediate release and timed-release, formulations. Examples of modified-release formulations include, for example, sustained-release (SR), extended-release (ER, XR, or XL), time-release or timed release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. In an embodiment, coatings that inhibit degradation of the compounds of the application by esterases, for example, plasma esterases, are used in the oral administration forms. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine, or phosphatidylcholines.

The dosage of compounds can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One skill in the art can determine the appropriate dosage based on the above factors. Compounds of the application may be administered initially in a suitable dosage that may be adjusted as required, depending on the clinical response. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily dose or the total daily dose may be divided into two, three, or four daily doses.

Compounds or compositions may be used alone or in combination with other known agents useful for treating diseases, disorders, or conditions related to oxidative stress and/or blood clotting. Compounds or compositions may also be used in combination with agents that inhibit esterases, such as plasma esterases. When used in combination with other agents useful in treating diseases, disorders, or conditions related to oxidative stress and/or blood clotting, it is an embodiment that the compounds or compositions are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. In a further embodiment, a combination of agents may be administered to a subject in a noncontemporaneous fashion. In certain embodiments, the other known agents may be clot-busting drugs, or anti-thrombolyic drugs. In a further embodiment, the anti-thrombolytic drug may be tPA. Treatment or prevention methods comprise administering to a subject or a cell, a therapeutically effective amount of the compounds or compositions, and optionally consists of a single administration, or alternatively comprises a series of administrations.

### The Methods

Having described the compounds generally, including the preferred compound candidates for use in the treatment of PD, the method utilized to test and produce the compounds may now be described. As noted, using the method of the present invention, more than one hundred compounds can be produced for use in the treatment of PD, but it has been found that there are four superior candidates for the treatment of PD. These candidates, which are referred to herein as RG-1, RG-2, RG-3, and RG-4, are found to be more stable and have more favorable characteristics in aqueous solutions and plasma.

The stability of these compounds is determined using a plasma stability assay and LC-MS/MS. The assays are utilized to assess each compound's activity and cellular permeability human dopaminergic neuronal precursor cell lines and Caco-2 cells. The synthesis of each of these candidates is outlined in 1-10 g batches (as described more fully below) and they demonstrate stability in solution and plasma at μM levels. A in silico analysis may be used to predict bioavailability, BBB penetration, and PK/PD profiles in the peripheral and central nervous system. Pharmacokinetics of the lead compound, maximum plasma concentration (nM to μM levels is expected) and brain uptake of three doses (>3-5% of plasma levels) is expected.

To determine the efficacy of the compounds, an α-synuclein (α-syn) fibril-injected mouse model for PD is utilized. Wild-type mice are injected with preformed α-syn fibrils to model PD and treated with top lead candidates. At the onset of PD pathologies, a dose-response study is performed to determine the optimal dose of the lead compound for the proof-of-concept and specific activity testing. To determine the efficacy of the lead compound in DA synthesis, protecting neurons, and reducing oxidative and inflammatory stresses, DA levels are measured in the striatum by HPLC and an assessment is made of the striatal TH expressing neurons, oxidative stress biomarker (malondialdehyde), gliosis, and α-syn deposits/aggregation. Based on the determined dose, the compound is compared with L-Dopa and non-treated mice for up to 24 weeks. The effectiveness of the compounds (particularly RG-1, RG-2, RG-3, and RG-4) are expected to be higher in DA levels (>1 nM) in the striatum of L-Dopa-treated mice compared to non-treated controls, higher levels of dopaminergic neurons, reduction in oxidative stress, gliosis, and α-syn aggregation and deposits. It is expected that these novel therapeutic agents can prevent the degeneration of DA neurons in an in vivo PD model and simultaneously enable dopaminergic neurons to synthesize DA.

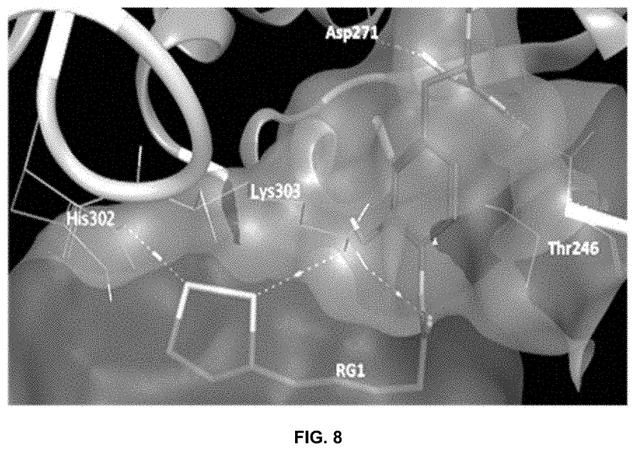

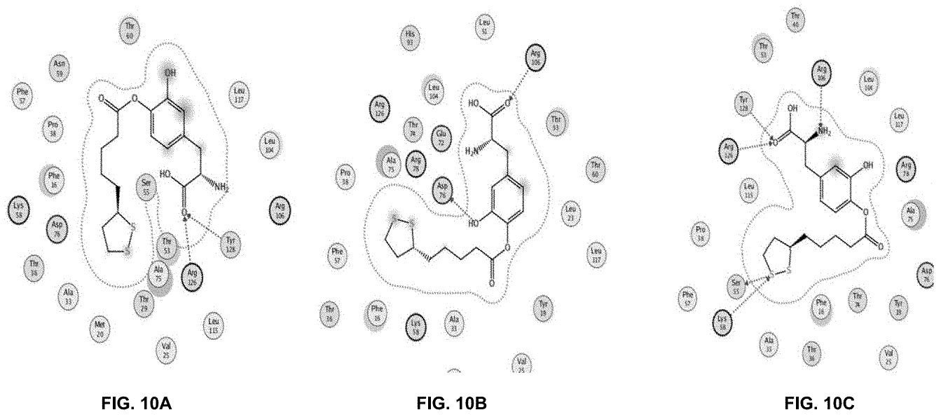

The design of the lead compounds (RG-1, RG-2, RG-3, and RG-4) begins with generating compounds with ALA and evaluating their behavior using a computer-aided drug design platform. A first evaluation is needed for, dopa-decarboxylase and α-syn binding sites followed by fatty acid transport proteins (FATP), and fatty acid binding proteins (FABP), binding sites. The compounds are modified based on size, polarity, conformational specificity, and their ability to generate stable salt bridges in the binding pockets of interest. After fragment-based drug design, machine learning models, docking, and a simulation-based platform to provide a list of compounds characterized as the lead compounds. These compounds are then subjected to simulation for ligand docking and molecular dynamic simulations on the binding sites of four critical targets; dopa-decarboxylase, α-syn, and FA transport proteins (FATP), and FA binding proteins (FABP). Using this method, it has been found that in dopa-decarboxylase case, RG-1 has strong binding interactions with the four key amino acid residues (namely Thr246, Asp271, His302, and Lys303) as illustrated in FIG. 8. Interactions with favorable affinity at the lowest energy levels are shown. Thr246 has H-acceptor and Asp271 has H-donor interactions with C═O and hydroxyl groups in carboxylic acid respectively. Among all amino acids, His302 has the strongest bond (H-acceptor, with −2.3 kcal/mol, 3.2 Å) with sulphur in dithiolane ring of ALA. Lys303 (a polar amino acid) has two H-acceptor bond interactions with ALA part of RG-1 at sulphur and oxygen atoms (FIG. 8). The predicted binding affinity of known dopa-decarboxylase inhibitors (carbidopa and benserazide), and L-Dopa itself were compared to RG-1. RG1-α-syn complex (in both glycerol and maltose binding sites) is positioned to form stable interaction with the residues Typ172, Ala169, and Met331 in its top five poses (FIGS. 10A (poses 1, 2 and 3), 11, and 12), suggesting the likelihood of target engagement of RG-1. At pose 1, Arg126 residue interacting (H-acceptor) with an oxygen atom in RG-1 with −1.0 kcal/mol energy at 3.24 Å distance. Likewise, Tyr128 residue interacting (H-acceptor) with an oxygen atom in RG-1 with −1.3 kcal/mol binding energy at 3.31 Å distance. In pose 2, Asp76 interacting (H-donor) with an oxygen atom in RG-1 with −1.6 kcal/mol energy at 2.70 Å distance. Likewise, Arg106 interacting (H-acceptor) with oxygen atom in RG-1 with −1.6 kcal/mol binding energy at 2.92 Å distance. In pose 3, Ser55 having an H-donor interaction with “S” atom in dithiolane with energy of −1.1 and 3.35 Å distance. Also, Arg126 and Arg106 with O and N atoms in RG-1 with energy of −3.6 and −1.1 kcal/mol and distances of 3.22 and 3.08 respectively. Further, “S” atom in RG-1 may also interact with Lys58 residue of FATP (H-acceptor bond) with a binding energy of −0.6 kcal/mol and distance of 4.14 Å. Both polar and non-polar residues (amino acids) are indicated by circles. Hydrogen bonding is indicated by dotted arrow. The proximity contour is the dotted line surrounding the RG-1. Dark shadows in of the residues indicate the receptor (FATP) exposure differences by the size and intensity of the quoits discs. The directions of the shadow indicate the directions of the amino acids towards the ligand (RG-1).

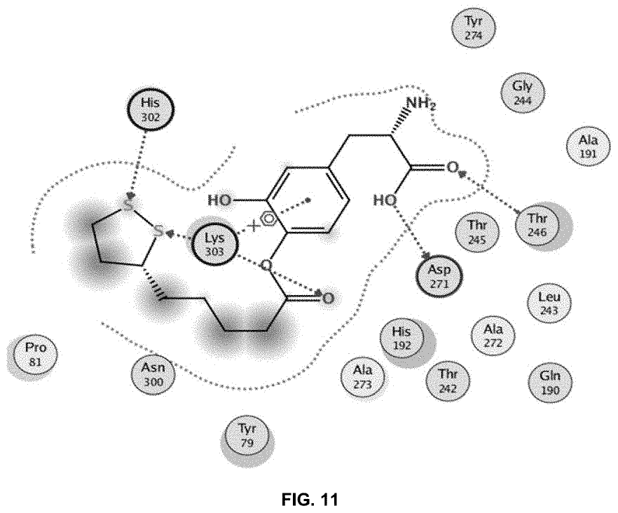

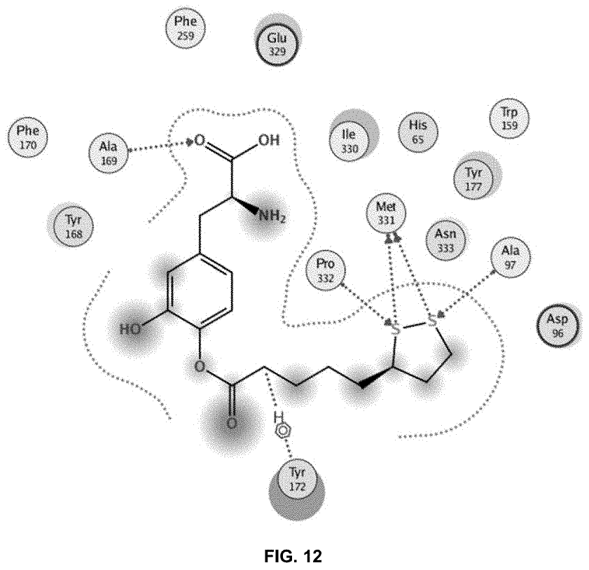

With reference to FIG. 11, RG1-Thr246 has the strongest binding energy (−2.5 kcal/mol) with an H-acceptor bond-type interaction with 3.19 Å with ═O of RG1. Lys303 can interact with the 6-ring of RG-1 with a pi-cation interaction bond with −0.7 kcal/mol binding energy. Likewise, Lys303 residue may interact with “S” or “0” atoms of RG-1. Asp271 residue interacting with hydroxy group of RG-1 (H-donor) with moderate binding energy of −1.7 kcal/mol and with a distance of 2.63 Å. His302 interacting with “S” atom in dithiolane with a weak energy of −0.6 kcal/mol. The clouds around the RG-1 atoms indicate that are exposed to the solvent. With reference to FIG. 12, Met331 residue can interact with either of with “S” atoms in 1,2-dithiolane with H-donor bonds and with the binding energies of −0.8 and −1.3 and with distances of 3.79 and 3.35 Å respectively. Pro332 and Ala169 and Ala97 residues can interact with RG-1 with H-acceptor bonds. Tyr172 residue can interact with the carbon atom of RG-1 (with an H-pi interaction bond) and with a binding energy of −0.5 kcal/mol (3.92 Å distance). Hydrogen bonding is indicated by dotted arrow, while arene-H interaction is shown by dotted line.

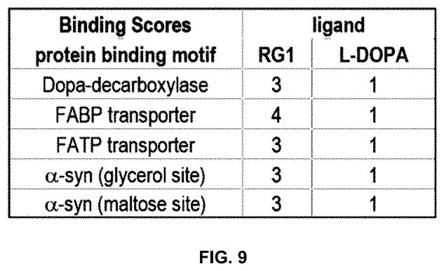

Top molecular docking poses are further validated with molecular dynamics simulation using GROMACS software on the 10-ns to 10-ms timescale. RG-1 in the periphery at a given therapeutic dose (e.g. −20-60 mg/kg) will be in an equilibrium state with dopa-decarboxylase. Our molecular dynamics simulation results demonstrated that both RG-1 and L-Dopa moderately interact (as substrate) with dopa-decarboxylate, while carbidopa block of dopa-decarboxylate activity was 4-5-fold stronger. It is well-established that carbidopa does not cross the BBB, and our molecular dynamics simulation (that began from the top docking poses) demonstrated that in the peripheral tissues, carbidopa binds to dopa-decarboxylate with a high affinity, and also this binding causes conformational changes that won't allow L-Dopa or RG-1 to bind to dopa-decarboxylate, hence, we consider carbidopa to be co-administrated with RG-1. It has also been found that RG-1 has predicted binding affinities in fatty acid transport proteins as shown in FIG. 9, as a virtual demonstration that RG-1 is capable of binding to FA transporters with high affinity, and then it will be transported through the membrane. FIG. 9 shows the binding scores for RG-1 compared to L-Dopa.

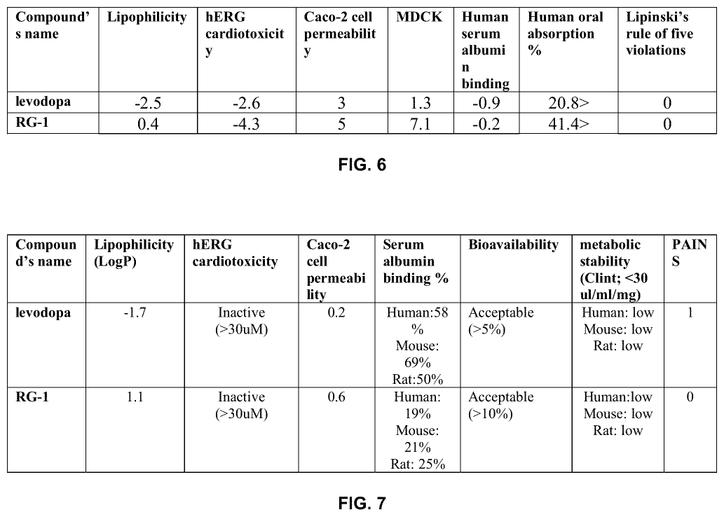

To further analyze the properties of the leading compounds (RG-1, RG-2, RG-3, and RG-4), the compounds are run through discovery modeling prediction platforms Schrodinger and ChemAxon to predict PK and ADME/tox properties of these compounds. When compared to L-Dopa, it is found that RG-1 is equal or superior to L-Dopa in all of the PK and ADME/tox parameters, as shown for example in FIGS. 6 and 7.

Lipophilicity is correlated to various models of drug properties affecting ADME/tox. Optimal lipophilicity ranges from 0-3. The predicted lipophilicity for L-Dopa is −2.51, which is similar to its determined experimental value −2.39 (Human Metabolome Database). RG-1 is less polar than L-Dopa with a predicted lipophilicity value of 0.4 (Schrödinger) and 1.1 (ChemAxon). IC₅₀ values for hERG are used as predictors of drug cardiotoxicity. Predicted IC₅₀ values for the blockage of hERG K⁺ channels are in a safe range for L-Dopa and RG-1. Schrödinger software even predicts that RG-1 has lower drug cardiotoxicity compared to L-Dopa. Caco-2 cell permeability is a determinant of intestinal absorption and oral bioavailability. Predicted permeability is considered low for both compounds, but RG-1 is predicted to have better permeability than L-Dopa by both Schrödinger and ChemAxon. This is supported by the higher percentage of human oral absorption predicted by Schrödinger (41.4% for RG-1 vs 20.8% for L-Dopa). MDCK cell permeability is considered a model for BBB penetration. Schrödinger software predicts RG-1 is 7 times more permeable than L-Dopa, as shown in FIG. 6. This supports Caco-2 cell permeability results, indicating RG-1 will have better bioavailability in the brain. Protein binding reduces free drug available to penetrate the brain to reach the therapeutic target. In both modeling software, the serum albumin binding for all species is lower in RG-1 compared to L-Dopa. This suggests RG-1 will have better bioavailability than L-Dopa, and further confirmed by ChemAxon bioavailability prediction.

Mutagenicity may be predicted using machine learning models using a series of AMES mutagenicity datasets and a rule-based mutagenicity filter. ChemAxon results allow for the determination that L-Dopa and RG-1 are non-mutagenic, as shown in FIG. 7. Lipinski's rule of 5 is used to evaluate drug likeness and determine whether the drug would likely be orally active in humans. No rules were violated for L-Dopa or RG-1, suggesting RG-1 would be orally active like L-Dopa. Pan-Assay Interference Compounds (PAINS) filters are used to filter out undesirable moieties and substructures that tend to react nonspecifically with numerous biological targets, rather than just the desired target. ChemAxon predicted no undesirable moieties for RG-1 but identified at least one violation for L-Dopa. Since the pharmacophore (the active moiety) of RG-1 exists in RG-2, RG-3, and RG-4, the inventors expect RG-2, RG-3, and RG-4 to behave similarly to RG-1.

Based on preliminary data, we have already screened more than 10,000 compounds in silico to identify 4 potential lead candidates with the best drug-like profiles and ADME/tox properties. We expect these 4 leads are suitable as brain-targeting drugs with additional favorable drug-like profiles and bioavailability in the brain in vitro and in vivo. We predict and expect significant efficacy in vivo PD mouse model in comparison with the current standard of care treatment, L-Dopa.

The caveats that virtual screen and computer based predictive indications employed are guides for the initial phase of drug development that required validation. The advantage of this approach is to increase the chance of success by taking advantage of in silico technologies that exist and are procured by RockGen. As an alternative approach to in silico, we will combine in silico studies and in vitro laboratory studies to address issues and pitfalls as they arise while characterizing the leads and testing in tissue culture systems. The PK studies proposed are standard type of studies essential for drug development and play an instrumental role in determining the drug-likeness and bioavailability, prediction of dose range for in vivo testing. The drug-like profiles and bioavailability in the brain in vitro and in vivo predict the readouts and study outcome carefully and uses alternative lead compound and adjusts our assay, if necessary, as a way to troubleshoot. One of the pitfalls that we are mindful of is the signs of adverse effects, if arise, lower the dose by 1-10 mg/kg increments suggested to be applied.

In the current standard of care treatment (L-Dopa), using in vivo models is expected to determine the efficacy in increasing DA levels and reducing neurodegeneration in an in vivo PD model, the α-synuclein fibril-injected mice (PFF mice). The findings from these studies are expected to be critical to support our rationale to propose for advancing the lead candidate for commercialization and towards the clinic for patients with PD.

Methods: There are availability of multiple mouse models for PD (alpha-sync fibril-injected mice, MPTP injected mice, 6-OHDA rats, rotenone injected rats, and transgenic mice (LRRK2 BAC, alpha-sync A153, thy1-hSNCA, Pitx3^{-/-}) to be utilized here. The chose the alpha-sync fibril-injected model is highly desirable because these mice replicate the behavioral and pathological features of majority of PD patients (sporadic PD) and the cause and the mechanism of PD due to α-syn is the most relevant to PD. Single intra-striatal inoculation of preformed fibrils (PFF) α-syn in wild-type nontransgenic mouse model widespread pathologic α-syn inclusions in the CNS that initiate Parkinson-like neurodegeneration and pathologies, progressive DA system degeneration, reduced DA levels culminating in motor deficits. Six-week old male C57Bl6 mice will be injected PFF α-syn at 5 μg, 2 mg/ml stereotactically on one side of the brain. Initially, a pilot cohort (n=4) will be injected with PFF to determine the time of onset of extend of pathologies resembling PD. Three cohorts A, B1 and B2 (n=8) for short term and dose finding studies and two cohorts C &D (n=12) for behavior and longer-term (12-24 weeks) pathological analysis will be established. At the onset of PD symptoms, we will perform a dose-response study with the lead candidate compound in cohort A, B1, and B2.

We have designed additional preclinical pharmacokinetics and most optimum route to deliver the top lead to the brain and area of α-syn pathology will be chosen by utilizing the data generated on the PK, plasma and brain levels. The i.t. delivery route will be an alternative and we would have the option to use it to assure the presence of the lead in the brain and α-syn pathology regions, which we are prepared for if warranted. Each of these methods of deliveries once selected, would have pitfalls to deal with and may positively or negatively impact the success of the proof-of-concept, therefore we will use data to select a suitable route. A cohort of α-syn fibril-injected mice will be treated with the top lead via p.o. (200 mg/kg/day). In case we use i.t. route, then we will deliver (10 mg/kg/day) via Alzet pump connected with cannula to deliver in area that α-syn PFF was inoculated and a cohort of vehicle-treated control mice will be used in comparison. A cohort of PFF inoculated mice will be treated with L-Dopa (200 mg/kg/day, p.o.) for comparison. In the third cohort of PFF inoculated mice, lead compound (objective dosing will be set to deliver up to 10 mM) will be treated via p.o. [or in case by i.t. via catheter connected osmotic pump (replaced every 28 days)] for 12-24 weeks. The proposed 12-24 week time will be sufficiently long enough to conduct behavioral studies (weight, 2× per week, wire-hang test and tapered balance beam, once at 12 or 24 weeks). To determine whether the top lead is a candidate for treating PD, we will measure DA levels in the striatum by HPLC compared to L-Dopa- and non-treated striatum. Additional validations, verifications, delivery route for the top lead will be addressed in a phase II study. The α-syn pathology in TH⁺ expressing neurons in SNpc will be evaluated and their progressive loss will be quantified using specific markers and neuronal counting. Due to PD-like Lewy pathology, SNpc neuron loss, reduced DA levels, we expect to observe significant behavioral abnormalities, e.g. poor performance on the wire-hang test and tapered beam walk.

inventors expect that DA levels in the striatum and substantia nigra tissues will reach equal to 0.5 to 1.2 ug/g of tissue, Upon that achievement the cohort C and D will be subjected to wire hang test and tapered beam walk to decipher treatment efficacy on the behavior vs vehicle treated control group. Additionally, the main objective and end results here will be to generate data on the lead compound exhibiting therapeutic and disease modifying effects. This will be quantified by assessing the TH⁺ neurons and staining with anti-tyrosine hydroxylase antibody (Abcam, Mass.) for the evidence of protected striatal TH⁺ expressing neurons, reduced gliosis using Iba-1 antibody (Abcam, Mass.), and blockage of α-syn inclusions/aggregation using anti-aggregated α-syn antibody clone 5G4 (Millipore, Mass.). The success and go milestone will be a statistically significant higher level of DA and higher TH⁺ neurons in α-syn PFF inoculated plus lead compound treated group as compared to α-syn PFF controls.

In this proposal, we are taking advantage of the guides from in silico prediction models followed by performing in an independent in vivo model for PD, the α-syn PFF injected wild type mouse system. We anticipate valuable and relevant data will be generated to validate the lead compounds for PD, that will be further developed by replicating and validation in alternative models of PD.

Unless otherwise stated, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein. It will be apparent to those skilled in the art that many more modifications are possible without departing from the inventive concepts herein.

Unless otherwise indicated, the definitions and embodiments described in this and other sections are intended to be applicable to all embodiments and aspects of the application herein described for which they are suitable as would be understood by a person skilled in the art. Terms of degree such as “about” and “approximately” as used herein mean a reasonable amount of deviation of the modified term such that the end result is not significantly changed. These terms of degree should be construed as including a deviation of at least +5% of the modified term if this deviation would not negate the meaning of the word it modifies. The term “derivative” as used herein refers to a compound that is derived from a parent compound by modification of one or more of the functional groups in the parent molecule. For example, a derivative of lipoic acid (LA) may be a reduced form (dithiol) of LA, or a reduced form in which the thiol groups are substituted with, for example, a C1-6 alkyl group or a C1-6 acyl group.

The term “subject” as used herein includes all members of the animal kingdom mammals, and suitably refers to humans. The term “pharmaceutically acceptable” means compatible with the treatment of subjects, in particular humans. The term “pharmaceutically acceptable salt” means an acid addition salt which is suitable for, or compatible with, the treatment of patients. The term “acid addition salt which is suitable for, or compatible with, the treatment of patients”, as used herein means any non-toxic organic or inorganic salt of any basic compound. Basic compounds that form an acid addition salt include, for example, compounds comprising a thiol group. Illustrative inorganic acids which form suitable salts include hydrochloric, hydrobromic, sulfuric, and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic, and salicylic acids, as well as sulfonic acids such as p-toluene sulfonic and methanesulfonic acids. Either the mono or di-acid salts can be formed, and such salts may exist in either a hydrated, solvated, or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The formation of the desired compound salt is achieved using standard techniques. For example, the basic compound is treated with an acid in a suitable solvent and the formed salt is isolated by filtration, extraction, or any other suitable method. The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

In embodiments of the described invention, compounds and compositions described herein have at least one asymmetric center. These compounds exist as enantiomers. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (e.g. less than 20%, suitably less than 10%, more suitably less than 5%) of compounds of the application having alternate stereochemistry. For example, compounds that are described or shown without any stereochemical designations are understood to be racemic mixtures. However, it is to be understood that all enantiomers and diastereomers are included within the scope of the present application, including mixtures thereof in any proportion. The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of the extent of disease, stabilized (i.e. not worsening) state of disease, preventing the spread of disease, delay or slowing of disease progression, amelioration or palliation of the disease state, diminishment of the reoccurrence of disease, and remission (whether partial or total), whether detectable or undetectable. “Treating” and “treatment” as used herein also include prophylactic treatment. For example, a subject can be treated to prevent onset or progression, or alternatively, a subject in post-stroke or post-infarct can be treated with a compound or composition as described herein to reduce injury or prevent recurrence. Treatment methods comprise administering to a subject a therapeutically effective amount of the compounds described. As used herein, the term “effective amount” or “therapeutically effective amount” means an amount effective, at dosages, and for periods of time necessary to achieve the desired result. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art.

In some embodiments, treatment with the compounds or compositions provided herein may be long-term treatments for chronic conditions, or maybe single-dose treatments for acute conditions. It will be appreciated that the embodiments described herein are for illustrative purposes, and not intended to be limiting in any way.

All terms used herein should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. When a Markush group or other grouping is used herein, all individual members of the group and all combinations and subcombinations possible of the group are intended to be individually included. All references cited herein are hereby incorporated by reference to the extent that there is no inconsistency with the disclosure of this specification. When a range is stated herein, the range is intended to include all sub-ranges within the range, as well as all individual points within the range. When “about,” “approximately,” or like terms are used herein, they are intended to include amounts, measurements, or the like that do not depart significantly from the expressly stated amount, measurement, or the like, such that the stated purpose of the apparatus or process is not lost.

The present invention has been described with reference to certain preferred and alternative embodiments that are intended to be exemplary only and not limiting to the full scope of the present invention, as set forth in the appended claims.

We claim:

1. A compound of Formula I: 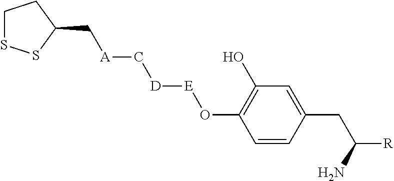
or a salt thereof, wherein:
A is chosen from methyl, ethyl, ethane, hydroxyl group, para chlorobenzene, benzenethiol, toluene, 4-methylphenol, phenyl, methanol, ethanol, methanol, ammonia, fluoromethane, nitrous acid, hydroxylamine, hydroxy(methyl)oxoammonium, trifluoromethane, chloromethane, chloride, sulphur, methanesulfinic acid, methanamine, ethanamine, nitromethane, hydroxylamine, and acetic acid;
C is chosen from methyl, Ethyl, ethane, carboxylic acid, hydroxyl group, para chlorobenzene, benzenethiol, toluene, 4-methylphenol, phenyl, methanol, ammonia, fluoromethane, trifluoroethane, nitrous acid, hydroxylamine, hydroxy(methyl)oxoammonium, chloromethane, chloride, sulphur, methanesulfinic acid, acetic acid, methanol, methanethiol, ethanol, ethenol, methanamine, and hydroxylamine;
D is chosen from methyl, ethyl, and methanethiol;
E is chosen from formaldehyde and ethyl; and
R is chosen from:

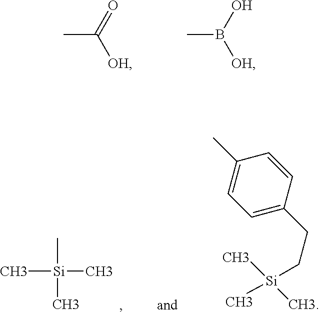

2. The compound of claim 1, wherein A is methyl, C is methyl, D is methyl, E is formaldehyde, and R is:

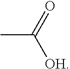

3. The compound of claim 1, wherein A is methyl, C is methyl, D is methyl, E is formaldehyde, and R is:

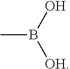

4. The compound of claim 1, wherein A is methyl, C is methyl, D is methyl, E is formaldehyde, and R is:

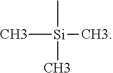

5. The compound of claim 1, wherein A is methyl, C is methyl, D is methyl, E is formaldehyde, and R is:

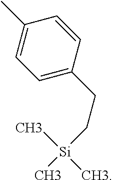

6. A compound of Formula I: 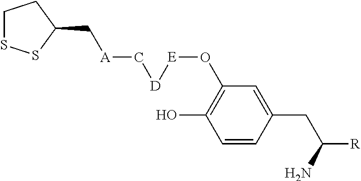
or a salt thereof, wherein:
A is chosen from methyl, ethyl, ethane, hydroxyl group, para chlorobenzene, benzenethiol, toluene, 4-methylphenol, phenyl, methanol, ethanol, methanol, ammonia, fluoromethane, nitrous acid, hydroxylamine, hydroxy(methyl)oxoammonium, trifluoromethane, chloromethane, chloride, sulphur, methanesulfinic acid, methanamine, ethanamine, nitromethane, hydroxylamine, and acetic acid;
C is chosen from methyl, Ethyl, ethane, carboxylic acid, hydroxyl group, para chlorobenzene, benzenethiol, toluene, 4-methylphenol, phenyl, methanol, ammonia, fluoromethane, trifluoroethane, nitrous acid, hydroxylamine, hydroxy(methyl)oxoammonium, chloromethane, chloride, sulphur, methanesulfinic acid, acetic acid, methanol, methanethiol, ethanol, ethenol, methanamine, and hydroxylamine;
D is chosen from methyl, ethyl, and methanethiol;
E is chosen from formaldehyde and ethyl; and
R is chosen from:

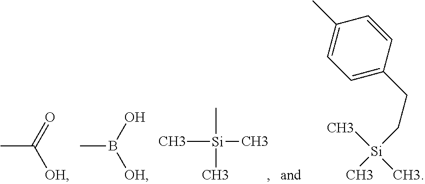

7. The compound of claim 6, wherein A is methyl, C is methyl, D is methyl, E is formaldehyde, and R is:

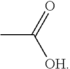

8. The compound of claim 6, wherein A is methyl, C is methyl, D is methyl, E is formaldehyde, and R is:

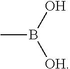

9. The compound of claim 6, wherein A is methyl, C is methyl, D is methyl, E is formaldehyde, and R is:

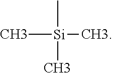

10. The compound of claim 6, wherein A is methyl, C is methyl, D is methyl, E is formaldehyde, and R is:

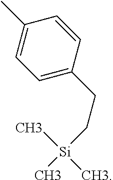

11. A method for treating Parkinson's Disease in a subject in need thereof, comprising administering to the subject a therapeutically effective amount of either of the following compounds: 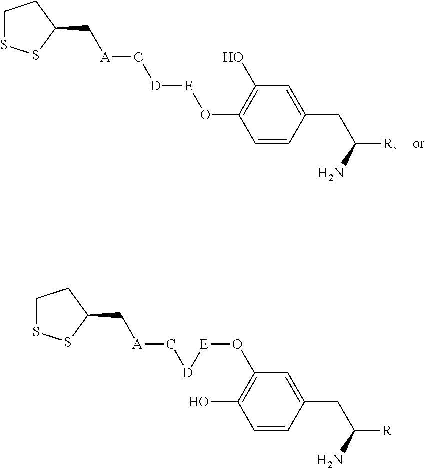
or a salt thereof, wherein:
A is chosen from methyl, ethyl, ethane, hydroxyl group, para chlorobenzene, benzenethiol, toluene, 4-methylphenol, phenyl, methanol, ethanol, methanol, ammonia, fluoromethane, nitrous acid, hydroxylamine, hydroxy(methyl)oxoammonium, trifluoromethane, chloromethane, chloride, sulphur, methanesulfinic acid, methanamine, ethanamine, nitromethane, hydroxylamine, and acetic acid;
C is chosen from methyl, Ethyl, ethane, carboxylic acid, hydroxyl group, para chlorobenzene, benzenethiol, toluene, 4-methylphenol, phenyl, methanol, ammonia, fluoromethane, trifluoroethane, nitrous acid, hydroxylamine, hydroxy(methyl)oxoammonium, chloromethane, chloride, sulphur, methanesulfinic acid, acetic acid, methanol, methanethiol, ethanol, ethenol, methanamine, and hydroxylamine;
D is chosen from methyl, ethyl, and methanethiol;
E is chosen from formaldehyde and ethyl; and
R is chosen from:

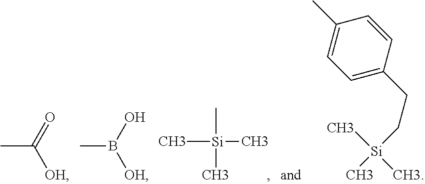

12. The method of claim 11, wherein A is methyl, C is methyl, D is methyl, E is formaldehyde, and R is:

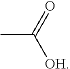

13. The method of claim 11, wherein A is methyl, C is methyl, D is methyl, E is formaldehyde, and R is:

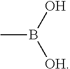

14. The method of claim 11, wherein A is methyl, C is methyl, D is methyl, E is formaldehyde, and R is:

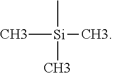

15. The method of claim 11, wherein A is methyl, C is methyl, D is methyl, E is formaldehyde, and R is: [chemical structure]
